(19) United States
(12) Patent Application Publication
(10) Pub. No.: US 2013/0017990 A1
(43) Pub. Date: Jan. 17, 2013

(54) LILIAL SURROGATE

(71) Applicant: Henkel AG & Co., KGaA, Duesseldorf (DE)

(72) Inventors: Ursula Huchel, Koln (DE); Ralf Bunn, Duesseldorf (DE); Manuela Materne, Kaarst (DE); Werner Faber, Willich (DE); Hubert Smyrek, Krefeld (DE); Theo ten Pierik, Venlo (NL); Frank Rittler, Duesseldorf (DE); Andreas Bauer, Kaarst (DE); Michael Dischmann, Krefeld (DE); Silvia Sauf, Duesseldorf (DE); Dagmar Preis-Amberger, Leverkusen (DE)

(73) Assignee: Henkel AG & Co., KGaA, Duesseldorf (DE)

(21) Appl. No.: 13/622,509

(22) Filed: Sep. 19, 2012

Related U.S. Application Data
(62) Division of application No. 13/232,339, filed on Sep. 14, 2011, now abandoned.
Application No. 13/232,339 is a continuation of application No. PCT/EP2010/051336, filed on Feb. 4, 2010.

(30) Foreign Application Priority Data
Mar. 16, 2009 (DE) 10 2009 001 570.1

(51) Int. Cl. A61L 9/01

(52) U.S. Cl. 512/10

(57) ABSTRACT

Due to toxicological concerns, it may be desirable to replace the fragrance compound lilial with less problematic compounds without losing the creative power and quality regarding perfumes. The present invention addresses this need by using selected oxazolidines such as silvialoxazolidine described herein in conjunction with 1 or more additional scent components selected from a group of compounds such as methoxymelonal.

### CROSS-REFERENCES TO RELATED APPLICATIONS

This application is a divisional application of U.S. patent application Ser. 13/232,339 filed on Sep. 14, 2011, which is a continuation of PCT/EP2010/051336, filed on Feb. 4, 2010, which claims priority under 35 U.S.C. §119 to DE 10 2009 001 570.1 filed on Mar. 16, 2009.

### FIELD OF THE INVENTION

The present invention generally relates to a scent composition with which toxicologically controversial lilial can be equivalently replaced. It furthermore relates to the use thereof to generate a lily-of-the-valley scent. It further relates to various agents, for example washing and cleaning agents, that encompass such a scent composition.

### BACKGROUND OF THE INVENTION

Many technical products and household products have scents added to them in order to mask their unpleasant inherent odor and/or to olfactorily upgrade said products or objects treated with them. In the case of washing and cleaning agents it is very desirable, in addition to scenting of the product itself, also to cover up unpleasant secondary odors from washing baths; in particular, however, scenting of the substrates to be treated is of great importance. Selection of the scents, and their composition, depends chiefly on the association that is to be evoked during utilization. In the case of all agents that have to do with washing or cleaning, for example, it may be advantageous to incorporate flowery notes into the composition, since they convey to a large number of consumers particularly positive emotions of springtime freshness and care provision. Flowery notes (floral notes) resp. scents (floral scents) are widespread in nature. Known examples are, for example, jasmine, lilac, rose, iris, or violet. The mixture of various floral notes is referred to in perfumery as a “bouquet.” With a bouquet of floral notes it also is possible, in particular, to emphasize the care-providing properties of an agent that is to be scented. One very popular floral scent is that of lilies-of-the-valley. A lily-of-the-valley scent with high acceptance among consumers and good stability in many products is lilial (CAS 80-54-6; 3-(4-tert-butylphenyl)-2-methylpropanal). Lilial is used in technical products, such as e.g. textile washing agents, as a scent, and generates a highly desired and pleasant lily-of-the-valley scent note. Lilial can also be readily incorporated into technical products such as, for example, washing and cleaning agents, and is stable in them over a wide pH range. It also possesses good substantivity, i.e. makes possible a long-lasting scent effect. According to recent findings, however, lilial may possibly have mutagenic potential, with the result that in accordance with EU classification criteria it could be classified as a category 3 mutagen (M:3). For CMR substances (CMR=carcinogenic, mutagenic, reprotoxic) in category 3, it is a requirement to demonstrate that the quantities used are harmless to consumers. Because of the toxicological misgivings, it is a matter of preventive and anticipatory consumer protection to replace this substance (lilial) with other, less problematic fragrances or scents.

Accordingly, it is desirable to provide a lilial surrogate, so that in typical product applications, for example in washing and cleaning agents, the use of lilial can be at least partly, but optimally entirely replaced, with no loss of perfume-making creativity and quality.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

The present invention provides an alternative for lilial so as to replace or at least greatly reduce the presence of this potentially problematic compound in fragrance compositions.

This present invention relates to a scent composition containing at least one oxazolidine in accordance with formula (I)

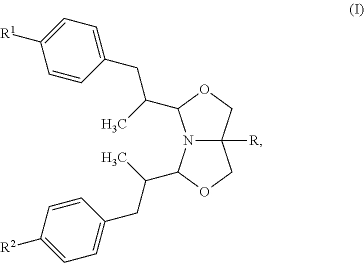

the residues R, R¹, R², mutually independently, denoting a hydrogen atom or an organic residue, in particular a linear or branched, substituted or unsubstituted alkyl or alkylene group from C₁ to C₂₀, it being preferred that residues R¹ and R² correspond to one another. Preferably, R═H or CH₃.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The scent composition can be made up entirely of one or more oxazolidines in accordance with formula (I). It is furthermore possible for the scent composition to contain, alongside at least one oxazolidine in accordance with formula (I), further fragrances as well. The terms “fragrance” and “scent” are used synonymously in the context of this invention. The scent composition according to the present invention contains by preference at least 0.01 wt %, advantageously at least 0.1 wt %, more advantageously at least 1 wt %, and in particular 5 wt % of one or more oxazolidines in accordance with formula (I). The minimum quantity of the oxazolidine in accordance with formula (I) in the scent composition according to the present invention can also be 10 wt %, 20 wt %, 30 wt %, 40 wt %, 50 wt %, 60 wt %, 70 wt % or, for example, also 80 wt %.

It is a particular advantage of this invention that the aforesaid scent composition can generate a particularly substantive and long-lasting lily-of-the-valley scent, in particular in conjunction with utilization in the field of textile laundering and care as well as in the cosmetic sector, but also in the so-called air-care sector. The aforesaid scent composition can be incorporated in stable fashion into the various compositions of textile laundering and care, and of the cosmetic sector and the air-care sector. The aforesaid scent composition is toxicologically more unproblematic than the lilial that is to be replaced. It is a further advantage of the invention that the scent composition according to the present invention, alone and also mixed with further scents, produces a scent-intensifying effect, i.e. the overall scent impression on the scented object becomes more intense and lasts longer.

In a preferred embodiment of the invention, an oxazolidine in accordance with formula (I) is contained in the scent composition according to the present invention, the two residues R¹ and R² in this formula each denoting an isobutyl residue [i.e. (CH₃)₂—CH—CH₂—], i.e. corresponding to an oxazolidine in accordance with formula (II)

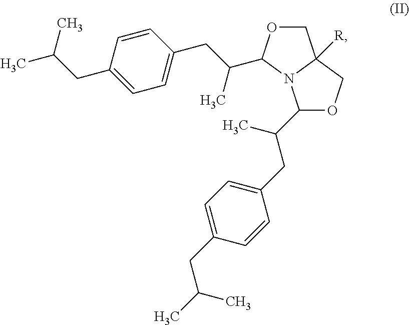

referred to here as silvialoxazolidine, contained by preference in quantities from 0.1 to 90 wt %, in particular from 1 to 40 wt %, the “wt %” being based on the entire quantity of the scent composition. In formula (II) as well, by preference R═H or CH₃. Scent compositions according to the present invention that contain silvialoxazolidine make possible particularly good results in terms of the desired replacement of lilial. In addition, they enable particularly long-lasting scent results, especially in textile laundering. In a preferred embodiment, the entire scent composition according to the present invention can be made up of silvialoxazolidine.

In a further preferred embodiment of the invention, an oxazolidine in accordance with formula (I) is contained in the scent composition according to the present invention, the two residues R¹ and R² in this formula each denoting an isopropyl residue [i.e. (CH₃)₂—CH—], corresponding to an oxazolidine in accordance with formula (III)

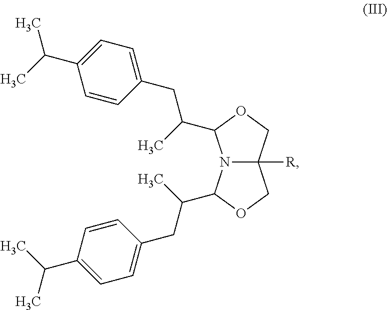

referred to here as cyclamoxazolidine, contained by preference in quantities from 0.1 to 90 wt %, in particular from 1 to 40 wt %, based on the entire quantity of the scent composition. In formula (III) as well, by preference R═H or CH₃. Scent compositions according to the present invention that contain cyclamoxazolidine make possible particularly good results in terms of the desired replacement of lilial, in particular in the application sector of washing or cleaning agents, especially with regard to pronounced substantivity of the desired scent impression during resp. subsequently to textile laundering. In a preferred embodiment, the entire scent composition according to the present invention can be made up of cyclamoxazolidine.

According to a further preferred embodiment of the invention, a preferred scent composition is notable for the fact that at least one further component is contained, selected from
(a) 6-methoxy-2,6-dimethylheptan-1-al (methoxymelonal) (by preference in quantities from 0.01 to 10 wt %, in particular from 0.01 to 5 wt %),
(b) 5,9-dimethyl-4,8-decadienal (geraldehyde) (by preference in quantities from 0.01 to 10 wt %, in particular from 0.01 to 5 wt %),
(c) beta-methyl-3-(1-methylethyl)benzenepropanal (florhydral) (by preference in quantities from 0.01 to 10 wt %, in particular from 0.01 to 5 wt %),
(d) 3-(4-tert-butylphenyl)propionaldehyde (bourgeonal) (by preference in quantities from 0.01 to 99 wt %, advantageously 0.01 to 20 wt %, in particular from 0.01 to 5 wt %),
(e) octahydro-8,8-dimethylnaphthalene-2-carbaldehyde (cyclomyral) (by preference in quantities from 0.01 to 10 wt %, in particular from 0.01 to 5 wt %),
(f) alpha-methyl-1,3-benzodioxole-5-propionaldehyde (helional) (by preference in quantities from 0.01 to 20 wt %, in particular from 0.01 to 5 wt %),
(g) 5-methyl-2-(1-methylbutyl)-5-propyl-1,3-dioxan (Troenan) (by preference in quantities from 0.01 to 10 wt %, in particular from 0.01 to 5 wt %),
(h) 3-(o-ethylphenyl)-2,2-dimethylpropionaldehyde (floralozone) (by preference in quantities from 0.01 to 10 wt %, in particular from 0.01 to 5 wt %),
(i) farnesol (by preference in quantities from 0.01 to 30 wt %, in particular from 0.01 to 5 wt %),
(j) 3,7,11-trimethyldodeca-1,6,10-trien-3-ol, by preference as an isomer mixture (nerolidol) (by preference in quantities from 0.01 to 50 wt %, in particular from 0.01 to 40 wt %),
(k) 2-methyl-4-phenylbutan-2-ol (dimethylphenylethylcarbinol) (by preference in quantities from 0.01 to 99 wt %, advantageously in quantities from 0.01 to 20 wt %, in particular from 0.01 to 15 wt %),
(l) cis-4-(isopropyl)cyclohexanemethanol (mayol) (by preference in quantities from 0.01 to 99 wt %, advantageously in quantities von 0.01 to 20 wt %, in particular from 0.5 to 15 wt %),
(m) 1-(1-hydroxyethyl)-4-(1-methylethyl)cyclohexane (by preference as a mixture of the diastereoisomers) (mugetanol) (by preference in quantities from 0.01 to 99 wt %, advantageously in quantities from 0.01 to 90 wt %, in particular from 0.5 to 40 wt %),
(n) (4-methyl-3-pentenyl)cyclohexenecarbaldehyde (citrusal) (by preference in quantities from 0.01 to 99 wt %, advantageously in quantities from 0.01 to 40 wt %, in particular from 0.1 to 20 wt %),
(o) cyclohexyl salicylate (by preference in quantities from 0.01 to 20 wt %, in particular from 0.1 to 10 wt %),
(p) hexyl salicylate (by preference in quantities from 0.01 to 20 wt %, in particular from 0.1 to 10 wt %),
(q) benzyl salicylate (by preference in quantities from 0.01 to 20 wt %, in particular from 0.1 to 10 wt %),
(r) amyl salicylate (by preference in quantities from 0.01 to 20 wt %, in particular from 1 to 10 wt %),
(s) 3-(p-(2-methylpropyl)phenyl)-2-methylpropionaldehyde (silvial) (by preference in quantities from 0.01 to 99 wt %, advantageously in quantities from 0.1 to 90 wt %, in particular from 1 to 40 wt %),
(t) 3-p-cumenyl-2-methylpropionaldehyde (cyclamenaldehyde) (by preference in quantities from 0.01 to 99 wt %, advantageously in quantities from 0.1 to 90 wt %, in particular from 1 to 40 wt %),
(u) mixture of: cis-tetrahydro-2-isobutyl-4-methylpyran-4-ol; trans-tetrahydro-2-isobutyl-4-methylpyran-4-ol; (florol) (by preference in quantities from 0.01 to 99 wt %, advantageously in quantities from 0.1 to 40 wt %, in particular from 1 to 40 wt %),
(v) triethyl citrate (by preference in quantities from 1 to 90 wt %, in particular from 5 to 80 wt %), and/or
(w) dipropylene glycol (by preference in quantities from 1 to 90 wt %, in particular from 5 to 80 wt %),
   the “wt %” being based in each case on the total quantity of the scent composition.

A scent composition according to the present invention that contains at least two, by preference at least four, advantageously at least eight, with further advantage at least 12, even more advantageously at least 16, in particular at least 20 components from the group of substances (a) to (w), corresponds to a preferred embodiment of the invention and makes possible a further improved lilial substitution.

A scent composition according to the present invention can, but need not, be limited to the ingredients recited above. Advantageously, the scent composition can also contain further usual constituents, for example essential oils, blossom oils, extracts from plant- and animal-based drugs, absolutes, resinoids, and fragrances isolated from natural products or chemically modified (semisynthetic) as well as those obtained entirely synthetically. The scent composition can advantageously also be diluted as desired using solvents, and stabilized with (an) emulsifier(s), to the extent that is desired or necessary.

A further subject of the present Application is a scent-comprising agent, in particular a washing or cleaning agent or cosmetic agent, that encompasses a scent composition according to the present invention. When an “agent according to the present invention” is discussed hereinafter, this refers, unless otherwise explained, to a scent-comprising agent of this kind that encompasses a scent composition according to the present invention, but in particular to a corresponding washing or cleaning agent or cosmetic agent. For purposes of this invention, a “scent” is any kind of fragrance, i.e. including flavors and essences, in particular those fragrances that trigger in humans a substantially positive odor perception.

According to a preferred embodiment, the scent-comprising agent is a textile treatment agent, an ironing adjuvant, a cleaning cloth, a washing agent, a cleaning agent, in particular for hard and/or soft surfaces, a household cleaner, a care-providing agent, a washing care-providing agent, a room scenting agent, an air improver, a conditioning agent, a coloring agent, a fabric softener, a conditioner substrate, a pharmaceutical, a plant protectant, a cleansing agent, a food, a cosmetic agent, a fertilizer, a construction material, an adhesive, a bleaching agent, a deliming agent, an automobile care agent, floor care agent, oven care agent, leather care agent, furniture care agent, a scrubbing agent, a disinfectant, a scenting agent, a mold removal agent, and/or a precursor product of the aforesaid agents.

Washing and cleaning agents in particular (especially textile washing agents or textile care agents, for example fabric softeners, but also cleaning agents for hard surfaces, for example a tile cleaner, bath cleaner, or sanitary cleaner), as well as any kind of care-providing agent, are particularly preferred, as are the air improvers and room scenting agents. Preferred cosmetic agents are by preference intended for skin care and/or hair care. Examples that could be recited are hair shampoo, cleaning lotion, shower gel, or bar soap. Most preferred, however, are any kinds of textile treatment agents, for example washing agents or fabric softeners, in both liquid and solid form.

According to a preferred embodiment, the scent-comprising agent comprises a weight proportion of the scent composition according to the present invention of ≧10⁻⁶ wt and ≦50 wt %, by preference ≧10⁻⁵ wt % and ≦40 wt %, preferably ≧10⁻⁴ wt % and ≦30 wt %, more preferably ≧10⁻³ wt % and ≦20 wt %, even more preferably ≧10⁻² wt % and ≦10 wt %, and most preferably ≧0.03 wt % and ≦5 wt %, based on the total weight of the agent.

According to a further preferred embodiment, the scent-comprising agent is present at least partly in solid form, in gel form, in foamed form, and/or in liquid form. If it is present in solid form, then preferably in the form of granulates, powders, shaped elements having at least one phase, and/or compressed shaped elements.

It is an advantage of the invention that the scent composition is very stable. Even in the various matrices and agents, for example cleaning agents or washing agents, the scent composition is stable and the scent of the scented agent is stable even when stored. A particular advantage of the scent compositions is their pronounced substantivity, which makes possible a particularly long-lasting scent effect.

According to a further preferred embodiment, the scent-comprising agent can additionally comprise, beyond the scent composition, at least one further scent, advantageously multiple further scents, by preference having a total weight concentration of these further scents of >0 wt % and <50 wt %, by preference ≧10⁻⁶ and ≦40 wt %, preferably 10⁻⁵ wt % and ≦30 wt %, more preferably 10⁻⁴ wt % and ≦20 wt %, even more preferably ≧10⁻³ wt % and ≦15 wt %, even more preferably ≧10⁻² wt % and ≦10 wt %, and most preferably ≧10⁴ wt % and ≦5 wt %, based on the total weight of the agent.

According to a further preferred embodiment, the additional scent(s) in the scent-comprising agent are selected from the group encompassing scents having scent notes of green notes, citrus notes, lavender notes, flowery notes, aldehyde notes, chypre notes, fougère notes, spice notes, oriental notes, wood notes, tobacco notes, and/or leather notes.

According to a further preferred embodiment, the additional scent(s) in the scent-comprising agent are selected from the group comprising scents of natural or synthetic origin, preferably more-volatile scents, higher-boiling scents, solid scents, and/or adherent scents.

It is a particular advantage of the invention that the scent composition according to the present invention, alone and also mixed with further scents, produces a scent-intensifying effect, i.e. the scent impression on the scented object becomes more intense and it lasts longer, especially when used in connection with textile washing or textile care.

Adherent fragrances that are usable in the context of the present invention are, for example, essential oils such as angelica oil, anise oil, arnica flower oil, basil oil, bay oil, bergamot oil, champaca flower oil, silver fir oil, silver fir cone oil, elemi oil, eucalyptus oil, fennel oil, fir needle oil, galbanum oil, geranium oil, gingergrass oil, guaiac wood oil, balsam gurjun oil, helichrysum oil, ho oil, ginger oil, iris oil, cajeput oil, calamus oil, chamomile oil, camphor oil, kanaga oil, cardamom oil, cassia oil, pine needle oil, balsam copaiva oil, coriander oil, curled peppermint oil, caraway oil, cumin oil, lavender oil, lemon grass oil, lime oil, tangerine oil, lemon balm oil, ambrette seed oil, myrrh oil, clove oil, neroli oil, niaouli oil, olibanum oil, orange oil, oregano oil, palmarosa oil, patchouli oil, balsam peru oil, petitgrain oil, pepper oil, peppermint oil, pimento oil, pine oil, rose oil, rosemary oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, ysop oil, cinnamon oil, cinnamon leaf oil, citronella oil, lemon oil, and cypress oil.

Higher-boiling resp. solid fragrances of natural or synthetic origin can, however, also be used advantageously in the context of the present invention as adherent fragrances resp. fragrance mixtures, i.e. scents. These compounds include the compounds recited below, as well as mixtures thereof: ambrettolide, <-amylcinnamaldehyde, anethole, anisealdehyde, anise alcohol, anisole, anthranilic acid methyl ester, acetophenone, benzyl acetone, benzaldehyde, benzoic acid ethyl ester, benzophenone, benzyl alcohol, benzyl acetate, benzyl benzoate, benzyl formate, benzyl valerate, borneol, bornyl acetate, <-bromostyrene, n-decylaldehyde, n-dodecylaldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, iron, isoeugenol, isoeugenol methyl ether, isosafrol, jasmone, camphor, carvacrol, carvone, p-cresol methyl ether, coumarin, p-methoxyacetophenone, methyl-n-amyl ketone, methylanthranilic acid methyl ester, p-methylacetophenone, methylchavicol, p-methylquinoline, methyl ®-naphthyl ketone, methyl-n-nonylacetaldehyde, methyl-n-nonyl ketone, muscone, ®-naphthol ethyl ether, ®-naphthol methyl ether, nerol, nitrobenzene, n-nonylaldehyde, nonyl alcohol, n-octylaldehyde, p-oxyacetophenone, pentadecanolide, ®-phenylethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymene, thymol, ©-undelactone, vanillin, veratrumaldehyde, cinnamaldehyde, cinnamyl alcohol, cinnamic acid, cinnamic acid ethyl ester, cinnamic acid benzyl ester.

Included among the more-volatile scents are, in particular, the lower-boiling fragrances of natural or synthetic origin that can be used alone or in mixtures. Examples of more-volatile fragrances are alkylsothiocyanates (alkylmustard oils), butanedione, limonene, linalool, linalyl acetate and propionate, menthol, menthone, methyl-n-heptenone, phellandrene, phenylacetaldehyde, terpinyl acetate, citral, citronellal.

All methods known in the existing art for the accelerated or delayed release of scents, in particular of the scent compositions according to the present invention, can be utilized, provided said methods appear to one skilled in the art to be suitable. According to a preferred embodiment of the invention, the agent according to the present invention encompasses supported and/or encapsulated scent, it being preferred that the scent composition according to the present invention is supported and/or encapsulated.

According to a further preferred embodiment, the scent-comprising agent, such as especially a washing, care-providing, or cleaning agent, comprises at least one, by preference multiple, active components, in particular washing-active, care-active, cleaning-active and/or cosmetic components, advantageously selected from the group encompassing anionic surfactants, cationic surfactants, amphoteric surfactants, nonionic surfactants, acidifying agents, alkalizing agents, anti-creasing compounds, antibacterial substances, antioxidants, antiredeposition agents, antistatic agents, builder substances, bleaching agents, bleach activators, bleach stabilizers, bleach catalysts, ironing adjuvants, cobuilders, scents, shrinkage preventers, electrolyes, enzymes, color protectants, coloring agents, dyes, color transfer inhibitors, fluorescent agents, fungicides, germicides, odor-complexing substances, adjuvants, hydrotropes, rinse aids, complexing agents, preservatives, corrosion inhibitors, optical brighteners, perfumes, perfume carriers, luster agents, pH adjusting agents, proofing and impregnation agents, polymers, swelling and anti-slip agents, foam inhibitors, sheet silicates, dirt-repelling substances, silver protectants, silicone oils, UV protection substances, viscosity regulators, thickening agents, discoloration inhibitors, graying inhibitors, vitamins, and/or fabric softeners.

For purposes of this invention, indications regarding the agent according to the present invention in wt % are based, unless otherwise indicated, on the total weight of the agent according to the present invention.

The quantities of the individual ingredients in the agents according to the present invention are based in each case on the intended use of the relevant agents, and one skilled in the art is familiar with the orders of magnitude of the quantities of ingredients to be used, or can gather them from the pertinent technical literature. The surfactant content selected, for example, will be greater or less depending on the intended use of the agents according to the present invention. For example, the surfactant content of, for example, washing agents, is between 10 and 50 wt %, by preference between 12.5 and 30 wt %, and in particular between 15 and 25 wt %, while cleaning agents for automatic dishwashing usually contain between 0.1 and 10 wt %, by preference between 0.5 and 7.5 wt %, and in particular between 1 and 5 wt % surfactants.

Washing or cleaning agents according to the present invention can contain, in particular, builder substances, surface-active surfactants, enzymes, bleaching agents, such as preferably organic and/or inorganic peroxygen compounds, peroxygen activators, water-miscible organic solvents, sequestering agents, electrolytes, pH regulators, thickeners, and further adjuvants such as soil release active substances, optical brighteners, graying inhibitors, color transfer inhibitors, foam regulators, and dyes.

The agents according to the present invention can contain surfactants; in particular anionic surfactants, nonionic surfactants, and mixtures thereof, but also cationic surfactants, are appropriate. Suitable nonionic surfactants are, in particular, ethoxylation and/or propoxylation products of alkyl glycosides and/or of linear or branched alcohols each having 12 to 18 carbon atoms in the alkyl portion and 3 to 20, by preference 4 to 10, alkyl ether groups. Also usable are corresponding ethoxylation and/or propoxylation products of N-alkylamines, vicinal diols, fatty acid esters and fatty acid amides that correspond, in terms of the alkyl portion, to the aforesaid long-chain alcohol derivatives, and of alkylphenols having 5 to 12 carbon atoms in the alkyl residue.

Suitable anionic surfactants are, in particular, soaps, and those that contain sulfate or sulfonate groups having preferably alkali ions as cations. Usable soaps are preferably the alkali salts of the saturated or unsaturated fatty acids having 12 to 18 carbon atoms. Such fatty acids can also be used in incompletely neutralized form. Included among the usable surfactants of the sulfate type are the salts of the sulfuric acid semiesters of fatty alcohols having 12 to 18 carbon atoms, and the sulfatization products of the aforesaid nonionic surfactants having a low degree of ethoxylation. Included among the usable surfactants of the sulfonate type are linear alkylbenzenesulfonates having 9 to 14 carbon atoms in the alkyl portion, alkanesulfonates having 12 to 18 carbon atoms, and olefinsulfonates having 12 to 18 carbon atoms that are produced upon reaction of corresponding monoolefins with sulfur trioxide, as well as alpha-sulfofatty acid esters that are produced upon sulfonation of fatty acid methyl or ethyl esters.

Cationic surfactants are by preference selected from among the esterquats and/or the quaternary ammonium compounds (QACs) in accordance with the general formula (R^{I})(R^{II})R^{III})R^{IV}) N⁺X⁻, in which R^{I} to R^{IV} denote identical or different C₁₋₂₂ alkyl residues, C₇₋₂₈ aralkyl residues, or heterocyclic residues, such that two or, in the case of an aromatic bond such as in pyridine, even three residues form, together with the nitrogen atom, the heterocycle (e.g. a pyridinium or imidazolinium compound), and X-denotes halide ions, sulfate ions, hydroxide ions, or similar anions. QACs are produced by the reaction of tertiary amines with alkylating agents such as, for example, methyl chloride, benzyl chloride, dimethyl sulfate, dodecyl bromide, but also ethylene oxide. The alkylation of tertiary amines having a long alkyl residue and two methyl groups occurs particularly easily, and the quaternization of tertiary amines having two long residues and one methyl group can also be carried out using methyl chloride under mild conditions. Amines that possess three long alkyl residues or hydroxy-substituted alkyl residues have low reactivity, and are quaternized, for example, using dimethyl sulfate. Suitable QACs are, for example, benzalkonium chloride (N-alkyl-N,N-dimethylbenzylammonium chloride), benzalkon B (m,p-dichlorobenzyldimethyl-C₁₂-alkylammonium chloride), benzoxonium chloride (benzyldodecyl-bis(2-hydroxyethyl)ammonium chloride), cetrimonium bromide (N-hexadecyl-N,N-trimethylammonium bromide), benzetonium chloride (N,N-dimethyl-N-[2-[2-[p-(1,1,3,3-tetramethylbutyl)phenoxy]ethoxy]ethyl]benzylammonium chloride), dialkyldimethylammonium chlorides such as di-n-decyldimethylammonium chloride, didecyldimethylammonium bromide, dioctyldimethylammonium chloride, 1-cetylpyridinium chloride, and thiazoline iodide, as well as mixtures thereof. Preferred QACs are the benzalkonium chlorides having C₈ to C₂₂ alkyl residues, in particular C₁₂ to C₁₄ alkylbenzyldimethylammonium chloride.

“Esterquats” are to be understood here as, by preference, compounds of the general formula IV,

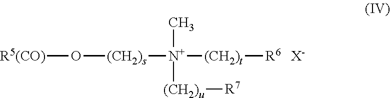

in which R⁵ denotes an alkyl or alkenyl residue having 12 to 22 carbon atoms and 0, 1, 2, or 3 double bonds; R⁶ and R⁷, mutually independently, denote H, OH, or O(CO)R⁵; s, t, and u, mutually independently in each case, denote the value 1, 2, or 3; and X⁻ denotes an anion, in particular halide, methosulfate, methophosphate, or phosphate, as well as mixtures thereof. Compounds that contain the group O(CO)R⁵ for R⁶, and an alkyl residue having 16 to 18 carbon atoms for R⁵, are preferred. Compounds in which R⁷ additionally denotes OH are particularly preferred. Examples of compounds of formula (IV) are methyl-N-(2-hydroxyethyl)-N,N-di(tallowacyloxyethyl)ammonium methosulfate, bis(palmitoyl)ethylhydroxyethylmethylammonium methosulfate, or methyl-N,N-bis(acyloxyethyl)-N-(2-hydroxyethyl)ammonium methosulfate. If quaternized compounds of formula (IV) that comprise unsaturated groups are used, those acyl groups whose corresponding fatty acids have an iodine number between 5 and 80, by preference between 10 and 60, and in particular between 15 and 45, and/or that have a cis/trans isomer ratio (in mol %) greater than 30:70, by preference greater than 50:50, and in particular greater than 70:30, are preferred. Commercially usual examples are the methylhydroxyalkyldialkoyloxyalkylammonium methosulfates marketed by the Stepan company under the trademark Stepantex®, or the products of Cognis Deutschland GmbH known under the trade name Dehyquat®, respectively the products of the manufacturer Goldschmidt-Witco known under the Rewoquat® designation.

Surfactants are contained in the washing agents according to the present invention in quantitative proportions of by preference 5 wt % to 50 wt %, in particular 8 wt % to 30 wt %. In laundry post-treatment agents in particular, by preference up to 30 wt %, in particular 5 wt % to 15 wt % surfactants, among them preferably at least in part cationic surfactants, are used.

An agent according to the present invention by preference contains at least one water-soluble and/or water-insoluble, organic and/or inorganic builder. Included among the water-soluble organic builder substances are polycarboxylic acids, in particular citric acid and sugar acids, monomeric and polymeric aminopolycarboxylic acids, in particular methylglycinediacetic acid, nitrilotriacetic acid, and ethylenediaminetetraacetic acid, as well as polyaspartic acid, polyphosphonic acids, in particular aminotris(methylenephosphonic acid), ethylenediaminetetrakis(methylenephosphonic acid), and 1-hydroxyethane-1,1-diphosphonic acid, polymeric hydroxy compounds such as dextrin, as well as polymeric (poly)carboxylic acids, polymeric acrylic acids, methacrylic acids, maleic acids, and mixed polymers thereof, which can also contain, polymerized into them, small proportions of polymerizable substances having no carboxylic-acid functionality. The relative molecular weight of the homopolymers of unsaturated carboxylic acids is generally between 5000 and 200,000, that of the copolymers between 2000 and 200,000, by preference 50,000 to 120,000, based in each case on free acid. A particularly preferred acrylic acid/maleic acid copolymer has a relative molecular weight from 50,000 to 100,000. Suitable (although less preferred) compounds of this class are copolymers of acrylic acid or methacrylic acid with vinyl ethers, such as vinylmethyl ethers, vinyl esters, ethylene, propylene, and styrene, in which the proportion of acid is equal to at least 50 wt %. It is also possible to use, as water-soluble organic builder substances, terpolymers that contain two unsaturated acids and/or salts thereof as monomers and, as a third monomer, vinyl alcohol and/or a vinyl alcohol derivative or a carbohydrate. The first acid monomer respectively salt thereof is derived from an ethylenically monounsaturated C₃ to C₈ carboxylic acid and by preference from a C₃ to C₄ monocarboxylic acid, in particular from (meth)acrylic acid. The second acid monomer respectively salt thereof can be a derivative of a C₄ to C₈ dicarboxylic acid, maleic acid being particularly preferred. The third monomeric unit is constituted in this case by vinyl alcohol and/or preferably an esterified vinyl alcohol. Vinyl alcohol derivatives that represent an ester of short-chain carboxylic acids, for example of C₁ to C₄ carboxylic acids, with vinyl alcohol, are particularly preferred. Preferred polymers contain 60 wt % to 95 wt %, in particular 70 wt % to 90 wt %, (meth)acrylic acid or (meth)acrylate, particularly preferably acrylic acid resp. acrylate, and maleic acid resp. maleinate, as well as 5 wt % to 40 wt %, by preference 10 wt % to 30 wt % vinyl alcohol and/or vinyl acetate. Very particularly preferred in this context are polymers in which the weight ratio of (meth)acrylic acid respectively (meth)acrylate to maleic acid respectively maleate is between 1:1 and 4:1, by preference between 2:1 and 3:1 and in particular 2:1 and 2.5:1. Both the quantities and the weight ratios are based on the acids. The second acid monomer respectively salt thereof can also be a derivative of an allylsulfonic acid that is substituted in the 2-position with an alkyl radical, by preference with a C₁ to C₄ alkyl radical, or with an aromatic radical that is derived by preference from benzene or benzene derivatives. Preferred terpolymers contain 40 wt % to 60 wt %, in particular 45 to 55 wt %, (meth)acrylic acid respectively (meth)acrylate, particularly preferably acrylic acid respectively acrylate, 10 wt % to 30 wt %, by preference 15 wt % to 25 wt % methallylsulfonic acid resp. methallylsulfonate, and as a third monomer 15 wt % to 40 wt %, by preference 20 wt % to 40 wt % of a carbohydrate. This carbohydrate can be, for example, a mono-, di-, oligo-, or polysaccharide, mono-, di-, or oligosaccharides being preferred. Sucrose is particularly preferred. The use of the third monomer is presumed to incorporate defined break points into the polymer, which are responsible for the polymer's good biodegradability. These terpolymers generally have a relative molecular weight between 1000 and 200,000, by preference between 200 and 50,000, and in particular between 3000 and 10,000. Further preferred copolymers are those that comprise, as monomers, acrolein and acrylic acid/acrylic acid salts, respectively vinyl acetate. Especially for the manufacture of liquid detergents, the organic builder substances can be used in the form of aqueous solutions, by preference in the form of 30- to 50-weight-percent aqueous solutions. All the aforesaid acids are used as a rule in the form of their water-soluble salts, in particular their alkali salts.

Organic builder substances\ can be contained if desired in quantities of up to 40 wt %, particularly up to 25 wt %, and particularly preferably from 1 wt % to 8 wt %. Quantities close to the aforesaid upper limit are used by preference in pasty or liquid, in particular water-containing, agents according to the present invention. Laundry post-treatment agents, for example fabric softeners, according to the present invention can also, if applicable, be free of organic builder.

Possibilities as water-soluble inorganic builder materials are, in particular, alkali silicates and polyphosphates, by preference sodium triphosphate. Crystalline or amorphous alkali aluminosilicates are used in particular as water-insoluble, water-dispersible inorganic builder materials, in quantities of e.g. up to 50 wt %, by preference not above 40 wt %, and in liquid agents in particular from 1 wt % to 5 wt %. Among these, the crystalline sodium aluminosilicates of washing-agent quality, in particular zeolite A, P, and if applicable X, are preferred. Quantities close to the aforesaid upper limit are used by preference in solid, particulate agents. Suitable aluminosilicates comprise, in particular, no particles having a particle size greater than 30 μm, and by preference are made up at a proportion of at least 80 wt % of particles having a size less than 10 μm.

Suitable substitutes respectively partial substitutes for the aforesaid aluminosilicate are crystalline alkali silicates, which can be present alone or mixed with amorphous silicates. The alkali silicates usable in the agents according to the present invention as detergency builders have by preference a molar ratio of alkali oxide to SiO₂ below 0.95, in particular from 1:1.1 to 1:12, and can be present in amorphous or crystalline fashion. Preferred alkali silicates are the sodium silicates, in particular the amorphous sodium silicates, having a Na₂O:SiO₂ molar ratio from 1:2 to 1:2.8. Crystalline sheet silicates of the general formula Na₂SiₓO₂ₓ₊₁.yH₂O, in which x, the so-called modulus, is a number from 1.9 to 4 and y is a number from 0 to 20, and preferred values for x are 2, 3, or 4, are preferred for use as crystalline silicates which can be present alone or in a mixture with amorphous silicates. Preferred crystalline sheet silicates are those in which x in the general formula recited assumes the values 2 or 3. In particular, both β- and δ-sodium disilicates (Na₂Si₂O₅.yH₂O) are preferred. Practically anhydrous crystalline alkali silicates manufactured from amorphous alkali silicates and having the aforesaid general formula, in which x denotes a number from 1.9 to 2.1, can also be used in agents according to the present invention. In a further preferred embodiment of agents according to the present invention, a crystalline sodium sheet silicate having a modulus from 2 to 3 can be used, such as the one that can be manufactured from sand and soda. Crystalline sodium silicates having a modulus in the range from 1.9 to 3.5 are used in a further preferred embodiment of agents according to the present invention. If alkali aluminosilicate, in particular zeolite, is present as an additional builder substance, the weight ratio of aluminosilicate to silicate, based in each case on anhydrous active substances, is equal to 1:10 to 10:1. In agents that contain both amorphous and crystalline alkali silicates, the weight ratio of amorphous alkali silicate to crystalline alkali silicate is by preference equal to 1:2 to 2:1, and in particular 1:1 to 2:1.

Builder substances are contained in the washing agents according to the present invention by preference in quantities of up to 60 wt %, in particular from 5 wt % to 40 wt %. Laundry post-treatment agents, for example fabric softeners, according to the present invention are by preference free of inorganic builders.

Peroxygen compounds that are suitable are, in particular, organic peracids respectively peracid salts of organic acids such as phthalimidopercapronic acid, perbenzoic acid, or salts of diperdodecanedioic acid, hydrogen peroxide, and inorganic salts that release hydrogen peroxide under application conditions, such as perborate, percarbonate, and/or persilicate. If solid peroxygen compounds are to be used, they can be utilized in the form of powders or granulates, which in principle can also be encased in known fashion. Alkali percarbonate, alkali perborate monohydrate, or (in particular in liquid agents) hydrogen peroxide in the form of aqueous solutions that contain 3 wt % to 10 wt % hydrogen peroxides, are used with particular preference, If a washing agent according to the present invention contains bleaching agents, such as preferably peroxygen compounds, the latter are present in quantities of preferably up to 50 wt %, in particular from 5 wt % to 30 wt %. The addition of small quantities of known bleaching-agent stabilizers, for example phosphonates, borates respectively metaborates, and metasilicates, as well as magnesium salts such as magnesium sulfate, may be useful.

Compounds that, under perhydrolysis conditions, yield aliphatic peroxocarboxylic acids having preferably 1 to 10 carbon atoms, in particular 2 to 4 carbon atoms, and/or (optionally substituted) perbenzoic acid, can be used as bleach activators. Substances that carry the O- and/or N-acyl groups having the aforesaid number of carbon atoms, and/or optionally substituted benzoyl groups, are suitable. Multiply acylated alkylenediamines, in particular tetraacetylethylendiamine (TAED), acylated triazine derivatives, in particular 1,5-diacetyl-2,4-dioxohexahydro-1,3,5-triazine (DADHT), acylated glycolurils, in particular tetraacetyl glycoluril (TAGU), N-acylimides, in particular N-nonanoyl succinimide (NOSI), acylated phenolsulfonates, in particular n-nonanoyl or isononanoyl oxybenzenesulfonate (n- or iso-NOBS), carboxylic acid anhydrides, in particular phthalic acid anhydride, acylated polyvalent alcohols, in particular triacetin, ethylene glycol diacetate, 2,5-diacetoxy-2,5-dihydrofuran, and enol esters, as well as acetylated sorbitol and mannitol respectively mixtures thereof (SORMAN), acylated sugar derivatives, in particular pentaacetylglucose (PAG), pentaacetylfructose, tetraacetylxylose and octaacetyllactose, as well as acetylated, optionally N-alkylated glutamine and gluconolactone, and/or N-acylated lactams, for example N-benzoylcaprolactam, are preferred. Hydrophilically substituted acyl acetates and acyl lactams are likewise used in preferred fashion. Combinations of conventional bleach activators can also be used. Such bleach activators can be contained in the usual quantity range, by preference in quantities from 1 wt % to 10 wt %, in particular 2 wt % to 8 wt %, based on the entire agent.

In addition to or instead of the aforementioned conventional bleach activators, sulfonimines and/or bleach-intensifying transition metal salts respectively transition metal complexes can also be contained as so-called bleach catalysts. Included among the appropriate transition metal compounds are, in particular, salen complexes of manganese, iron, cobalt, ruthenium, or molybdenum and nitrogen-analog compounds thereof, carbonyl complexes of manganese, iron, cobalt, ruthenium, or molybdenum, complexes of manganese, iron, cobalt, ruthenium, molybdenum, titanium, vanadium, and copper having nitrogen-containing tripod ligands, ammine complexes of cobalt, iron, copper, and ruthenium. Combinations of bleach activators and transition metal bleach catalysts can likewise be used. Bleach-intensifying transition metal complexes, in particular having the central atoms Mn, Fe, Co, Cu, Mo, V, Ti, and/or Ru, can be used in usual quantities, by preference in a quantity of up to 1 wt %, in particular from 0.0025 wt % to 0.25 wt %, and particularly preferably from 0.01 wt % to 0.1 wt %, based in each case on the entire agent.

Suitable enzymes usable in the agents are those from the class of the proteases, cutinases, amylases, pullulanases, hemicellulases, cellulases, lipases, oxidases, and peroxidases, as well as mixtures thereof. Enzymatic active substances recovered from fungi or bacteria, such as Bacillus subtilis, Bacillus licheniformis, Streptomyces griseus, Humicola lanuginosa, Humicola insolens, Pseudomonas pseudoalcaligenes, or Pseudomonas cepacia, are particularly suitable. The enzymes that are used as applicable can be adsorbed onto carrier substances and/or embedded into encasing substances in order to protect them from premature inactivation. They are contained in the washing agents according to the present invention by preference not above 5 wt %, in particular from 0.2 wt % to 2 wt %.

The agents can contain as optical brighteners, for example, derivatives of diaminostilbenedisulfonic acid or alkali metal salts thereof. Suitable, for example, are salts of 4,4′-bis(2-anilino-4-morpholino-1,3,5-triazinyl-6-amino)stilbene-2,2′-disulfonic acid, or compounds of similar structure that carry, instead of the morpholino group, a diethanolamino group, a methylamino group, an anilino group, or a 2-methoxyethylamino group. Brighteners of the substituted diphenylstyryl type can also be present, e.g. the alkali salts of 4,4′-bis(2-sulfostyryl)diphenyl, of 4,4′-bis(4-chloro-3-sulfostyryl)diphenyl, or of 4-(4-chlorostyryl)-4′-(2-sulfostyryl)diphenyl. Mixtures of the aforesaid optical brighteners can also be used.

Included among the suitable foam inhibitors are, for example, organopolysiloxanes and mixtures thereof with microfine, optionally silanated silicic acid, as well as paraffin waxes and mixtures thereof with silanated silicic acid or bis-fatty acid alkylenediamides. Mixtures of different foam inhibitors, for example those made of silicones, paraffins, or waxes, are also used with advantage. The foam inhibitors, in particular silicone- and/or paraffin-containing foam inhibitors, are by preference bound to a granular carrier substance that is soluble respectively dispersible in water. Mixtures of paraffins and bistearylethylenediamide are particularly preferred in this context.

In addition, the agents can also contain components (so-called “soil release” active substances) that positively influence the ability of oils and fats to be washed out of textiles. This effect becomes particularly apparent when the soiled textile is one that has already been previously washed several times with a washing agent according to the present invention that contains this oil- and fat-releasing component. The preferred oil- and fat-releasing components include, for example, nonionic cellulose ethers such as methyl cellulose and methylhydroxypropyl cellulose having a 15 to 30 wt % proportion of methoxy groups and a 1 to 15 wt % proportion of hydroxypropoxyl groups, based in each case on the nonionic cellulose ethers, as well as polymers, known from the existing art, of phthalic acid and/or terephthalic acid resp. of their derivatives with monomeric and/or polymeric diols, in particular polymers of ethylene terephthalates and/or polyethylene glycol terephthalates or anionically and/or nonionically modified derivatives thereof.

The agents can also contain color transfer inhibitors, by preference in quantities from 0.1 wt % to 2 wt %, in particular 0.1 wt % to 1 wt %, which in a preferred embodiment of the invention are polymers of vinylpyrrolidone, vinylimidazole, vinylpyridine-N-oxide, or copolymers thereof. Also usable are both polyvinylpyrrolidones having molecular weights from 15,000 to 50,000 and polyvinylpyrrolidones having molecular weights above 1,000,000, in particular from 1,500,000 to 4,000,000, N-vinylimidazole/N-vinylpyrrolidone copolymers, polyvinyloxazolidones, copolymers based on vinyl monomers and carboxylic acid amides, pyrrolidone-group-containing polyesters and polyamides, grafted polyamidoamines and polyethylenimines, polymers having amide groups made up of secondary amines, polyamine-N-oxide polymers, polyvinyl alcohols, and copolymers based on acrylamidoalkenyl sulfonic acids. It is also possible, however, to use enzymatic systems encompassing a peroxidase and hydrogen peroxide respectively a substance that yields hydrogen peroxide in water. The addition of a mediator compound for the peroxidase, for example an acetosyringone, a phenol derivative, or a phenothiazine or phenoxazine, is preferred in this case, in which context aforementioned polymeric color transfer-inhibiting active substances can also be additionally used. Polyvinylpyrrolidone has by preference, for use in agents according to the present invention, an average molar weight in the range from 10,000 to 60,000, in particular in the range from 25,000 to 50,000. Among the copolymers, those of vinylpyrrolidone and vinylimidazole at a molar ratio from 5:1 to 1:1, having an average molar weight in the range from 5000 to 50,000, in particular from 10,000 to 20,000, are preferred.

The purpose of graying inhibitors is to keep dirt that has been detached from the textile fibers suspended in the bath. Water-soluble colloids, usually organic in nature, are suitable for this, for example starch, size, gelatin, salts of ethercarboxylic or ethersulfonic acids of starch or of cellulose, or salts of acid sulfuric acid esters of cellulose or of starch. Water-soluble polyamides containing acid groups are also suitable for this purpose. Starch derivatives other than those recited above can also be used, for example aldehyde starches. Cellulose ethers such as carboxymethyl cellulose (sodium salt), methyl cellulose, hydroxyalkyl cellulose, and mixed ethers such as methylhydroxyethyl cellulose, methylhydroxypropyl cellulose, methylcarboxymethyl cellulose, and mixtures thereof are preferably used, for example in quantities from 0.1 to 5 wt % based on the agent.

Included among the organic solvents usable in the agents according to the present invention, especially when the latter exist in liquid or pasty form, are alcohols having 1 to 4 carbon atoms, in particular methanol, ethanol, isopropanol, and tert-butanol, diols having 2 to 4 carbon atoms, in particular ethylene glycol and propylene glycol, as well as mixtures thereof, and the ethers derivable from the aforesaid compound classes. Water-miscible solvents of this kind are present in the washing agents according to the present invention by preference in quantities not above 30 wt %, in particular from 6 wt % to 20 wt %.

In order to establish a desired pH that does not result of itself from mixture of the other components, the agents according to the present invention can contain system-compatible and environmentally compatible acids, in particular citric acid, acetic acid, tartaric acid, malic acid, lactic acid, glycolic acid, succinic acid, glutaric acid, and/or adipic acid, but also mineral acids, in particular sulfuric acid, or bases, in particular ammonium hydroxides or alkali hydroxides. pH regulators of this kind are contained in the agents according to the present invention in quantities preferably not above 20 wt %, in particular from 1.2 wt % to 17 wt %.

The manufacture of solid agents according to the present invention presents no difficulties and can in principle occur in known fashion, for example by spray-drying or granulation; the peroxygen compound and bleach catalyst can, if applicable, be added later. The scent composition according to the present invention, as well as optionally further fragrances, are by preference applied onto the agent at the end of the manufacturing operation. A method comprising an extrusion step is preferred for the manufacture of agents according to the present invention having an elevated bulk weight, in particular in the range from 650 g/l to 950 g/l. The manufacture of liquid agents according to the present invention likewise presents no difficulties and can likewise occur in known fashion, the scent composition, as well as optionally further fragrances, being by preference introduced into the agent at the end of the manufacturing operation.

According to a preferred embodiment, the teaching of the present invention can be used to significantly decrease the perfume proportion in washing, cleaning, and body-care agents. It is thereby possible to offer perfumed products even for those particularly sensitive consumers who, because of specific incompatibilities and irritations, can use normally perfumed products in only limited fashion or not at all. Chiefly to be mentioned in this connection are skin-care products and deodorants, but also washing agents such as, for example, hand washing agents.

A preferred solid, in particular powdered, washing agent according to the present invention can by preference contain, alongside the scent composition according to the present invention, components that are selected, for example, from the following:
anionic surfactants such as, by preference, alkylbenzenesulfonate, alkyl sulfate, e.g. in quantities by preference from 5 to 30 wt %,
nonionic surfactants such as, by preference, fatty alcohol polyglycol ethers, alkylpolyglucoside, fatty acid glucamide, e.g. in quantities by preference from 0.5 to 15 wt %,
detergency builders such as, for example, zeolite, polycarboxylate, sodium citrate, in quantities from, for example, 0 to 70 wt %, advantageously 5 to 60 wt %, by preference 10 to 55 wt %, in particular 15 to 40 wt %,
alkalis such as, for example, sodium carbonate, in quantities e.g. from 0 to 35 wt %, advantageously 1 to 30 wt %, by preference 2 to 25 wt %, in particular 5 to 20 wt %,
bleaching agents such as, for example, sodium perborate, sodium percarbonate, in quantities e.g. from 0 to 30 wt %, advantageously 5 to 25 wt %, by preference 10 to 20 wt %,
corrosion inhibitors, e.g. sodium silicate, in quantities e.g. from 0 to 10 wt %, advantageously 1 to 6 wt %, by preference 2 to 5 wt %, in particular 3 to 4 wt %,
stabilizers, e.g. phosphonates, advantageously 0 to 1 wt %,
foam inhibitor, e.g. soap, silicone oils, paraffins, advantageously 0 to 4 wt %, by preference 0.1 to 3 wt %, in particular 0.2 to 1 wt %,
enzymes, e.g. proteases, amylases, cellulases, lipases, advantageously 0 to 2 wt %, by preference 0.2 to 1 wt %, in particular 0.3 to 0.8 wt %,
graying inhibitor, e.g. carboxymethyl cellulose, advantageously 0 to 1 wt %,
discoloration inhibitor, e.g. polyvinylpyrrolidone derivatives, advantageously 0 to 2 wt %,
adjusting agent, e.g. sodium sulfate, advantageously 0 to 20 wt %,
optical brightener, e.g. stilbene derivative, biphenyl derivative, advantageously 0 to 0.4 wt %, in particular 0.1 to 0.3 wt %,
optionally further fragrances,
optionally water,
optionally soap,
optionally bleach activators,
optionally cellulose derivatives,
optionally dirt repellents,
   “wt %” being based in each case on the entire agent.

In another preferred embodiment of the invention, the washing or cleaning agent is present in liquid form, by preference in gel form. Preferred liquid washing or cleaning agents have water contents of, for example, 10 to 95 wt %, by preference 20 to 80 wt %, and in particular 30 to 70 wt %, based on the entire agent. In the case of liquid concentrates the water content can also be particularly low, e.g. <30 wt %, by preference <20 wt %, in particular <15 wt %, “wt %” being based in each case on the entire agent. The liquid agents can also contain non-aqueous solvents.

A preferred liquid, in particular gel-type, washing agent according to the present invention can by preference contain, alongside the scent composition according to the present invention, components that are selected e.g. from the following:
anionic surfactants such as, by preference, alkylbenzenesulfonate, alkyl sulfate, e.g. in quantities by preference from 5 to 40 wt %,
nonionic surfactants such as, by preference, fatty alcohol polyglycol ethers, alkylpolyglucoside, fatty acid glucamide, for example in quantities by preference from 0.5 to 25 wt %,
detergency builders such as, for example, zeolite, polycarboxylate, sodium citrate, advantageously 0 to 15 wt %, by preference 0.01 to 10 wt %, in particular 0.1 to 5 wt %,
foam inhibitor, e.g. soap, silicone oils, paraffins, in quantities e.g. from 0 to 10 wt %, advantageously 0.1 to 4 wt %, by preference 0.2 to 2 wt %, in particular 1 to 3 wt %,
enzymes, e.g. proteases, amylases, cellulases, lipases, in quantities e.g. from 0 to 3 wt %, advantageously 0.1 to 2 wt %, by preference 0.2 to 1 wt %, in particular 0.3 to 0.8 wt %,
optical brightener, e.g. stilbene derivative, biphenyl derivative, in quantities e.g. from 0 to 1 wt %, advantageously 0.1 to 0.3 wt %, in particular 0.1 to 0.4 wt %,
optionally further fragrances,
optionally stabilizers,
water,
optionally soap, in quantities e.g. from 0 to 25 wt %, advantageously 1 to 20 wt %, by preference 2 to 15 wt %, in particular 5 to 10 wt %,
optionally solvent (by preference alcohols), advantageously 0 to 25 wt %, by preference 1 to 20 wt %, in particular 2 to 15 wt %,
   “wt %” being based in each case on the entire agent.

A preferred liquid fabric softener according to the present invention can contain by preference, alongside the scent composition according to the present invention, components that are selected from the following:
cationic surfactants, such as especially esterquats, e.g. in quantities from 5 to 30 wt %,
cosurfactants such as, for example, glycerol monostearate, stearic acid, fatty alcohols, fatty alcohol ethoxylates, e.g. in quantities from 0 to 5 wt %, by preference 0.1 to 4 wt %,
emulsifiers such as, for example, fatty amine ethoxylates, e.g. in quantities from 0 to 4 wt %, by preference 0.1 to 3 wt %,
optionally further scents,
optionally dyes, by preference in the ppm range,
optionally stabilizers, by preference in the ppm range,
solvents such as, in particular, water, in quantities by preference from 60 to 90 wt %,
   “wt %” being based in each case on the entire agent.

A further subject of the invention is the use of a scent composition according the present invention to generate a lily-of-the-valley fragrance, in particular in washing, cleaning, or body-care applications.

A further subject of the invention is the use of a scent composition according to the present invention to substitute for lilial, in particular in washing or cleaning agents and in cosmetic agents.

A further subject of the invention is the use of cyclamoxazolidine to substitute for lilial in scent compositions or perfumed agents, in particular in scent-containing washing or cleaning agents and in scent-containing cosmetic agents, by preference in order to generate a lily-of-the-valley scent that is more substantive as compared with lilial, in particular in washing, cleaning, or body-care applications.

A further subject of the invention is the use of silvialoxazolidine to substitute for lilial in scent compositions or perfumed agents, in particular in scent-containing washing or cleaning agents and in scent-containing cosmetic agents, by preference in order to generate a lily-of-the-valley fragrance that is more substantive as compared with lilial, in particular in washing, cleaning, or body-care applications.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A scent composition containing at least one oxazolidine in accordance with formula (I) 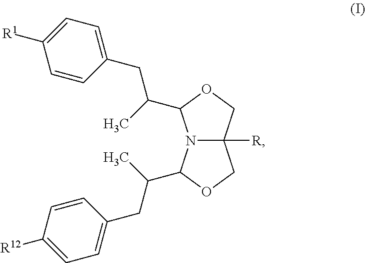
wherein the residues R, R¹, R² each are independently selected from the group consisting of a hydrogen atom or a linear or branched, substituted or unsubstituted alkyl or alkylene group from C₁ to C₂₀; and wherein the scent composition comprises at least one further component, selected from the group consisting of:
(a) 6-methoxy-2,6-dimethylheptan-1-al (methoxymelonal) in quantities from 0.01 to 10 wt %;
(b) 5,9-dimethyl-4,8-decadienal (geraldehyde) in quantities from 0.01 to 10 wt %;
(c) beta-methyl-3-(1-methylethyl)benzenepropanal (florhydral) in quantities from 0.01 to 10 wt %;
(d) 3-(4-tert-butylphenyl)propionaldehyde (bourgeonal) in quantities from 0.01 to 99 wt %;
(e) octahydro-8,8-dimethylnaphthalene-2-carbaldehyde (cyclomyral) in quantities from 0.01 to 10 wt %;
(f) alpha-methyl-1,3-benzodioxole-5-propionaldehyde (helional) in quantities from 0.01 to 20 wt %;
(g) 5-methyl-2-(1-methylbutyl)-5-propyl-1,3-dioxan (Troenan) in quantities from 0.01 to 10 wt %;
(h) 3-(o-ethylphenyl)-2,2-dimethylpropionaldehyde (floralozone) in quantities from 0.01 to 10 wt %;
(i) farnesol in quantities from 0.01 to 30 wt %;
(j) 3,7,11-trimethyldodeca-1,6,10-trien-3-ol as an isomer mixture (nerolidol) in quantities from 0.01 to 50 wt %;
(k) 2-methyl-4-phenylbutan-2-ol (dimethylphenylethylcarbinol) in quantities from 0.01 to 99 wt %;
(l) cis-4-(isopropyl)cyclohexanemethanol (mayol) in quantities from 0.01 to 99 wt %;
(m) 1-(1-hydroxyethyl)-4-(1-methylethyl)cyclohexane as a mixture of the diastereoisomers (mugetanol) in quantities from 0.01 to 99 wt %;
(n) (4-methyl-3-pentenyl)cyclohexenecarbaldehyde (citrusal) in quantities from 0.01 to 99 wt %;
(o) cyclohexyl salicylate in quantities from 0.01 to 20 wt %;
(p) hexyl salicylate in quantities from 0.01 to 20 wt %;
(q) benzyl salicylate in quantities from 0.01 to 20 wt %;
(r) amyl salicylate in quantities from 0.01 to 20 wt %;
(s) 3-(p-(2-methylpropyl)phenyl)-2-methylpropionaldehyde (silvial) in quantities from 0.01 to 99 wt %;
(t) 3-p-cumenyl-2-methylpropionaldehyde (cyclamenaldehyde) in quantities from 0.01 to 99 wt %;
(u) mixture of: cis-tetrahydro-2-isobutyl-4-methylpyran-4-ol; trans-tetrahydro-2-isobutyl-4-methylpyran-4-ol; (florol) in quantities from 0.01 to 99 wt %;
(v) triethyl citrate in quantities from 1 to 90 wt %; and
(w) dipropylene glycol in quantities from 1 to 90 wt %,
the “wt %” being based in each case on the total quantity of the scent composition.

2. The scent composition according to claim 1 wherein the composition contains at least two components from the group (a) to (w).

3. The scent composition according to claim 1 wherein the composition contains at least four components from the group (a) to (w).

4. The scent composition according to claim 1 wherein the composition contains at least eight components from the group (a) to (w).

5. The scent composition according to claim 1 wherein the composition contains at least twelve components from the group (a) to (w).

6. The scent composition according to claim 1 wherein the composition contains at least sixteen components from the group (a) to (w).

7. The scent composition according to claim 1 wherein the composition contains at least twenty components from the group (a) to (w).
